(19) United States
(12) Patent Application Publication
(10) Pub. No.: US 2017/0333511 A1
(43) Pub. Date: Nov. 23, 2017

(54) COMPOSITION COMPRISING HYALURONIC ACID AND ROC® AND ITS USE

(71) Applicant: EYE TECH S.R.L., Genova (IT)

(72) Inventor: Giuseppe CAMPORA, Ruta di Camogli (IT)

(21) Appl. No.: 15/544,097

(22) PCT Filed: May 7, 2015
(86) PCT No.: PCT/EP2015/060040

§ 371 Date: Jul. 17, 2017

(30) Foreign Application Priority Data
Jan. 27, 2015 (IT) MI2015A000095

(51) Int. Cl. A61K 36/752; A61K 8/36; A61K 8/73; A61K 31/728; A61K 8/9789; A61K 9/00; A61K 47/12

(52) U.S. Cl. CPC A61K 36/752; A61K 9/0095; A61K 8/36; A61K 47/12; A61K 31/728; A61K 8/9789; A61K 9/0053; A61K 8/735; A61K 2800/524; A61K 2800/92

(57) ABSTRACT

A composition containing hyaluronic acid and a Vitamin C containing extract obtained from pigmented varieties of blood oranges and its use is described.

The present invention relates to a composition comprising hyaluronic acid and Red Orange Complex (ROC®).

The present invention relates also to the medical and/or cosmetic use of said composition, preferably in rheumatology, dermatology, ophthalmology and gastroenterology.

Nowadays, the request of products that can effectively fight the different diseases and problems related to oxidative stress phenomena is ever more increasing; this is caused by a numbers of factors among which it is recalled the increasing of the average life together with a particular lifestyle and a greater exposure to polluting agents. Therefore, the request of products that can act as anti-inflammatories, antioxidants, immunomodulators and neuro-hepato-cardio protectors is always greater.

At the state of art some products of this type already exist; an example is ascorbic acid or vitamin C.

Vitamin C is a dietetic element essential for many cell and tissue processes such as the biosynthesis of collagen; it is furthermore essential as a co-factor in the biosynthesis of cathecolamines, L-carnitine, cholesterol, amino acids and it is potentially involved in the prevention of cardiovascular diseases and cancer.

The deficiency of vitamin C causes many pathological conditions among which scurvy with fragility of blood capillaries and damage of the connective tissue. Vitamin C is also known to be a powerful antioxidant, that inhibits the peroxidation of the lipid membranes and acts as a scavenger against free radicals. A dietary supplement of vitamin C improves the performances of the immune system such as the antimicrobial and natural killer activity and increases the lymphocytic proliferation. Moreover, it protects against the damages caused by UV counteracting the negative effects caused by radiation.

The daily administration of vitamin C is associated to an improvement of the joint functions, a reduction of the bone marrow's fractures and of the sizes of bones, important pathological conditions in the pathogenesis of osteoarthritis. The role of vitamin C in ocular processes is not less important. The two main antioxidants of the eye are ascorbic acid and glutathione (GSH) that are present in aqueous humor. While all the tissues contain high levels of these antioxidants, the corneal epithelium has the highest concentration of ascorbic acid and the lens has the highest concentration of GSH, pointing out that this two antioxidants are particularly important for the protection of those tissues from oxidative stress.

Vitamin C can be defined as a physiological “sun protection” due to its high concentrations in the aqueous humor and its ability to absorb the UV light as it prevents the penetration of UV light and protects the tissues from light-induced oxidative damage.

Many mammals have the ability to synthesize vitamin C de novo; however the human beings and few other species do not have the necessary enzyme for its synthesis and therefore they are based exclusively on its assumption through the diet.

Another product with antioxidant effect which also comprises vitamin C is ROC®. It is a standardized extract obtained from the juice and peels of three pigmented varieties of blood orange (Moro, Tarocco and Sanguinello) grown exclusively in a particular area surrounding the Etna volcano in Sicily (Italy). These oranges grow in a territory which is particularly subjected to strong thermal excursions and then they keep inside them a high antioxidant power as a defense from the external environment.

The ROC®is an extract containing a specific blend of active principles:
1. anthocyanins 3.1% (cyanidin-3-glucoside);
2. hydroxycinnamic acids (caffeic, cumaric, ferulic, sinapic acid) 2.1%;
3. flavanones (hesperidin, narirutin) 8.1%;
4. ascorbic acid 5%.
   1 Anthocyanins (AC): ROC® has a high concentration of AC. AC are natural water-soluble pigments and are found in various plants. Despite their limited oral bioavailability and the low plasma concentrations, the use of these pigments has been associated to an important protection against different human pathological conditions among which cardiovascular diseases. Many studies have underlined that some benefits of AC are due to their position, particularly, at endothelial level, contributing to vascular homeostasis, to the control of angiogenesis, platelet inflammation and aggregation, thank to their ability to modulate the expression and the activity of numerous enzymes involved in the metabolism of NO. Moreover, scientific studies report that AC can prevent the expression of adhesion molecules and pro-inflammatory agents.
   2—Hydroxycinnamic acids: a group of molecule that are normally present in the food such as caffeic, cumaric, ferulic and sinapic acid with important therapeutic activities (for example they reduce the risk of developing diabetes type 2 (DM2) or Alzheimer disease and have anti-inflammatory properties (Shin Ds and coll) belongs to the class of hydroxycinnamic acids. Cumaric acid was evaluated for its immunomodulatory and anti-inflammatory properties in vivo, showing itself as a potential immunosupprensive agent in the treatment of immune anti-inflammatory diseases such as rheumatoid arthritis.
   3—Flavanones have a protective activity of blood vessels in addition to an anti aetheromatosis action also due to the suspension of the HMG-coenzyme A reductase enzyme activity (which plays a key role in the synthesis of cholesterol in the human body and thus influence its plasma levels). The inhibition of its activity decreases the concentrations of intracellular cholesterol and generates the consequent increase of the expression of LDL receptors. This, in its turn, increases the cell picking of LDL lipoproteins and the removal of cholesterol from the circulation. Hesperidin is a good example of flavanone which can be found in lemons and oranges, that reduces the level of cholesterol in blood by acting on the HMG-CoA enzyme, improves the endothelial function and reduces the inflammation and affects the lipid profile in patients suffering from metabolic syndrome. It has been demonstrated that such flavanones, such as rutin (vitamin P) and its derivatives, together with hesperidin, help the seal and strengthen the walls of blood vessels. These substances, in a similar way to vitamin C, are able to improve the synthesis of collagen and to make then the connective tissue of blood vessels more elastic.
   4—Ascorbic acid: the presence of ascorbic acid or vitamin C, a dietary element essential for many cell and tissue processes, whose characteristics are above described, is primary in ROC®. All the elements that are present in ROC® work together increasing the antioxidant and anti-free-radical ability. For example, anthocyanins are pigments which protect against the capillary weakness and against many ageing processes or cellular modifications caused by oxygen, among which inflammatory processes and carcinogenic modifications.

In fact, the action of ROC® as antioxidant effectiveness was greater than that of vitamin C only and of resveratrol (extracted from the grape acinus) in delaying the skin ageing due to an excessive exposure to sunlight and many environmental pollutants. Moreover, the mixture of antioxidants can be very useful to people subjected to strong oxidative stress such as sportsmen, diabetics and smokers. For example, the dietary supplementation with ROC® reduces the risk which is associated with pathological conditions, such as diabetes type I, without any side effect and prevents patients from complications related to this disease that are partially caused by lipid liperoxidation by modulating the key molecules involved in the inflammatory process. The effectiveness of ROC® can be attributed not only to the content of anthocyanins but also to the phytophenol levels and the structural characteristics of the phytochemicals contained in it in addition to their ability to interact with biomolecules.

These characteristics lead to the conclusion that a daily supplementary of ROC® can be an additional protection against oxidation in vivo of cellular biomolecules.

Another dietetic supplement marketed and used against oxidative processes is hyaluronic acid (HA). HA is chemically defined as a glycosaminoglycan (GAG) constituted by repeated subunits of disaccharides N-acetylglucosamine and glucuronic acid. HA is distributed everywhere throughout the tissues and animal fluids, at high concentrations in synovial fluids, in vitreous humor and in the skin and it is the main responsible for the viscosity and the lubricant activity of the synovial fluid; in the cartilage HA acts as a support for the aggregation of proteoglycans and proteins. Moreover, HA represents an important regulator of the cell functions, as demonstrated by the presence of receptors both for hyaluronic acid and its fragments on the cell membrane. Hyaluronic acid is usually marketed as a salt (hyaluronate sodium).

The use of hyaluronic acid as a dietary supplement has spread after a series of experimental evidences which have highlighted its ability of prevent from age-related degenerative processes such as articular diseases.

The main difference in the biological activity of HA is related to its molecular weight. Defined molecular weights are attributed to the lubricant properties, to intracellular signals and probably to the effectiveness of intra-articular injections of HA for the treatment of osteoarthritis.

The molecular weight around 500000 Da seems to be the limit for the different properties related to hyaluronic acid; in fact:
higher molecular weights are mostly associated to HA benefits, while
lower molecular weights have a different or an opposite activity with respect to the native fragments with high molecular weight. The fragments with very low molecular weight are not able to bind the receptors that are present on the cell membrane or to transduce the signals into the cell and it has been demonstrated to be associated with the stimulation of neovascularization, migration and metastases of the cancerous cells and to be lacking of anti-inflammatory activity or have a pro-inflammatory activity with respect to the native molecule in many biological systems.

The dietary supplements more available on the market are substantially of three types (Bucci L. R. et al, J. Appl. Nutrition, 2004, 54 10-33 (1)):
HA from sternal cartilage of chicken, obtained through enzymatic degradation of collagen II, which has a low molecular weight (50-10000 Da);
HA from the cockscomb, obtained through enzymatic denaturation, which has a molecular weight comprises from 50000 to 200000 Da and are used for injections;
HA from bacterial fragmentation (Streptococcus) with a high molecular weight.

The third type of HA is essentially a purified HA. The molecular weight is around a million Dalton and exactly corresponds to the native hyaluronic acid that is present in the cartilage and in the synovial fluids. In this preparation, the proteins are absent or present in traces. As this type of HA for food use is purified by companies that produce HA for pharmaceutical use, it is more certain that these products have controlled quality standards, toxicological and reproducibility tests with respect to the previously described types of HA.

HA with a molecular weight of 1 million Dalton increases the viscosity, has lubricant properties and anti-inflammatory activity. In fact, one of the mechanisms proposed for the intra-articular injections of HA in patients affected by osteoarthritis is the normalization of intra-cellular signals of the synovium by the molecules of HA with high molecular weight.

One of the main prerequisites for any beneficial effect of the dietary supplements containing hyaluronic acid is the absorption with the consequent release at the tissue level. Only one, among the three types of the mentioned hyaluronic acid, the purified HA, has experimental evidences of oral absorption (Shauss A G at al., FASEB J 2004 Apr; 18(4), A150-A151, Balogh L et al.).

The Applicant has now surprisingly found that a composition comprising hyaluronic acid and ROC® shows unexpected synergisms.

The present invention relates in fact to a pharmaceutical or cosmetic composition comprising hyaluronic acid and ROC®.

Preferably, said composition comprises preservatives.

Preferably, said preservatives are selected from the group consisting of potassium sorbate, sodium benzoate.

Preferably, in said composition hyaluronic acid has a molecular weight higher than 500000 Da. More preferably said hyaluronic acid has a molecular weight of 1000000 Da.

According to a preferred embodiment of the invention, hyaluronic acid is obtained by bacterial fermentation.

Preferably, the composition of the present invention comprises (percentages expressed in weight/final weight):
hyaluronic acid in the range of 50-55%;
ROC® in the range of 35-45%;
preservatives in the range of 0-12%.

More preferably the composition of the present invention comprises hyaluronic acid at 52%, ROC® at 38.40% and preservatives at 9.60%.

In a preferred embodiment, the composition of the present invention is liquid and for oral use. Said liquid composition comprises (% expressed in weight/total weight)
water in the range of 97-99%,
hyaluronic acid in the range of 0.5-1%,
ROC® in the range of 0.5-0.7%
preservatives in the range of 0-0.2%

More preferably said liquid composition comprises:
water at 98.28%,
hyaluronic acid at 0.90%,
ROC® at 0.66%,
preservatives at 0.16%.

In a preferred embodiment the composition of the present invention is for topic use.

The composition of the present invention can be realized according to the following steps:
1. In an aqueous solution mix hyaluronic acid with high molecular weight and ROC®and heat at 95° C. for 30 minutes.
2. Cool down the solution obtained from 1. to 65° C. and add the preservatives:

potassium sorbate and sodium benzoate.
3. Cool down at 20° C. and make the quality checks. The final formulation provides a composition with hyaluronic acid 9 mg/ml and ROC® 6.66 mg/ml.

The present invention further relates to the medical and/or cosmetic use of the herein described composition. Preferably the present invention relates to the use of the herein described composition in rheumatology, dermatology, ophthalmology and gastroenterology.

Therefore, the present invention is based on the synergic association of pure hyaluronic acid with high molecular weight (1 million Da) from bacterial fragmentation and ROC® to react against pathological and/or physiological age-related degenerative processes. As previously described, they both have regenerative, antioxidant and antiinflammatory characteristics being essential cell constituents.

The herein described synergic association has moreover the aim to advantageously develop a product that is easy to use, being liquid and for oral use, that can bring essential nutrients to improve the bone and cartilage function, the visual function, the skin regeneration and protection against damages caused by UV rays as well as to act against age-related degenerative processes. Particularly, to combine hyaluronic acid with ROC® having a high concentration of anthocyanins, hydroxycinnamic acids (caffeic, cumaric, ferulic, sinapic acid), flavanones (hesperidin, narirutin) and vitamin C, which are elements of natural origin and therefore easy to be absorbed by the body. These elements have together an anti-inflammatory, immunomodulator, antioxidant, neuroprotective, hepatoprotective and cardioprotective function.

Advantageously, the composition of the present invention is able to perform different and synergic activities suitable for the use in rheumatology, dermatology, ophthalmology and gastroenterology. The formulation of HA and ROC® allows, in a single product, to give a supply of: anthocyanins with an anti-inflammatory and antioxidant activity, hydroxycinnamic acids with an immunomodulatory activity, flavanones with a protective activity on blood vessels, ascorbic acid which is an essential element for the cell functions.

In ROC® the elements work in sinergy resulting more effective compared to the single elements. In fact, ROC® has shown to have a greater antioxidant action than vitamin C.

Moreover, ROC® is a standardized extract of tarocco oranges. The single elements contained in ROC® are of natural origin and not of chemical synthesis with safe absorption by the body.

In addition, HA with high molecular weight from bacterial fermentation is purified and standardized. Scientific evidences show that hyaluronic acid with a 1 million Da weight is able to be absorbed by the body and to distribute itself where necessary.

Moreover, vitamin P or Rutin that is present in ROC® allows to this composition to have also a protective effect on the wall of blood vessels with its antiradical activity and the prevention of platelet aggregation. In fact, Rutin inhibits the activation of phospholipase C causing, through a chain reaction, the inhibition of platelet aggregation.

Some representative examples of the possible realizations of the invention, without considering them limitative, are below reported:

### Example 1

1. Hyaluronic acid with a high molecular weight (9 mg/ml) and ROC® (6.66 mg/ml) were mixed in an aqueous solution and the water was heated at 95° C. for 30 minutes.
2. The resultant solution from 1. was cooled down to 65° C. and, once it was reached the said temperature, the preservatives potassium sorbate (1 mg/ml-0.10%) and sodium benzoate (0.6 mg/ml-0.07%) were added.
3. The resultant solution from 2. was subsequently cooled down to 20° C.
4. Quality checks were made on the resultant solution from 3.
The final formulation is a liquid composition which comprises (% expressed in weight/final weight):
Water: 98.28%
Hyaluronic acid: (9 mg/ml): 0.90%
ROC (6.66 mg/ml): 0.66%
Preservatives: 0.16%

1. A pharmaceutical or cosmetic composition comprising hyaluronic acid and ROC®.

2. The composition according claim 1, wherein said composition comprises preservatives.

3. The composition according claim 2, wherein said preservatives are selected from the group consisting of potassium sorbate, and sodium benzoate.

4. The composition according to claim 1, wherein said hyaluronic acid has a molecular weight higher than 500000 Da.

5. The composition according to claim 1, wherein said hyaluronic acid has a molecular weight of 1000000 Da.

6. The composition according to claim 1, wherein said composition comprises:
hyaluronic acid in the range of 50-55%;
ROC in the range of 35-45%;
preservatives in the range of 0-12%.

7. The composition according to claim 6, wherein said composition comprises:
hyaluronic acid at 52.00%;
ROC® at 38.40%;
preservatives at 9.60%.

8. The composition according to claim 1 wherein said composition is liquid and for oral use.

9. (canceled)

10. (canceled)
